(19) United States
(12) Patent Application Publication
(10) Pub. No.: US 2021/0116459 A1
(43) Pub. Date: Apr. 22, 2021

(54) BIOMARKERS AND USES THEREOF

(71) Applicant: Oxford University Innovation Limited, Oxford (GB)

(72) Inventors: Manu VATISH, Oxford (GB); Christopher REDMAN, Oxford (GB); Ian SARGENT, Oxford (GB); Adam CRIBBS, Oxford (GB); Gavin COLLETT, Oxford (GB); Wei ZHANG, Oxford (GB)

(21) Appl. No.: 17/046,786

(22) PCT Filed: Apr. 11, 2019
(86) PCT No.: PCT/GB2019/051054

§ 371 Date: Oct. 11, 2020

(30) Foreign Application Priority Data
Apr. 12, 2018 (GB) 1806042.6

(51) Int. Cl. G01N 33/68; A61K 31/616

(52) U.S. Cl. CPC G01N 33/689; A61K 31/616; G01N 2333/575; G01N 2333/78; G01N 2800/50; G01N 2333/705; G01N 2800/368; G01N 2800/56; G01N 2800/52; G01N 2333/96486

(57) ABSTRACT

A method of determining the pre-eclampsia status of a pregnant subject, comprising providing a biological sample obtained from the subject; and determining the presence or absence, and/or level, of one or more of Type XVII collagen alpha 1, leptin, neprilysin, Filamin B and Scavenger receptor class B member 1 in the biological sample, and preferably comparing the level determined with a control.

The present invention relates to novel biomarkers for preeclampsia, and to uses of the novel biomarkers.

Preeclampsia is a disorder of pregnancy characterized by pregnancy-induced hypertension and proteinuria, which can lead to eclampsia (convulsions), and other serious maternal and/or foetal complications. Preeclampsia is originated in early gestation from the failure of implantation mechanisms and/or placental development, and is thus closely related to complications of pregnancy in early gestation such as including but not limited to implantation failure, and threatened and spontaneous miscarriage. Preeclampsia affects approximately 5-7% of pregnant women (approximately 8,370,000 pregnant women worldwide per year) and is a major cause of maternal and perinatal mortality. Furthermore, women with preeclampsia have an 8-fold higher risk of cardiovascular death later in their life, and offspring born from pregnancies affected by preeclampsia have an increased risk of metabolic and cardiovascular disease and mortality later in life. In addition to the medical complications suffered by mothers and risks to their children, preeclampsia is also a huge financial burden to healthcare systems worldwide.

Currently preeclampsia is diagnosed if a pregnant woman, with previously normal blood pressures, presents with new-onset hypertension coupled with proteinuria that develops after 20 weeks of gestation.

Accordingly, there is a need for a reliable test for preeclampsia, preferably a predictive test, which allows intervention early in pregnancy to avoid complications and allows for careful monitoring during pregnancy. If preeclampsia is diagnosed before 16 weeks gestation the mother can be prescribed aspirin which can reduce the occurrence of preeclampsia by about 50 to 60%. An aim of this invention is to provide such a test, which may be undertaken at between about 10 and about 12 weeks of pregnancy. The present invention provides one or more novel biomarkers and biomarker combinations that allow for the prediction and/or early detection/diagnosis of preeclampsia.

Accordingly, in a first aspect of the invention there is provided a method of determining the preeclampsia status of a pregnant subject, comprising:
(a) providing a biological sample obtained from the subject; and
(b) determining the presence or absence, and/or level, of one or more of Type XVII collagen alpha 1, leptin, neprilysin, Filamin B and Scavenger receptor class B member 1 in the biological sample.

Preferably, in step (b), at least the presence, absence and/or level of Type XVII collagen is determined.

The term ‘preeclampsia status’ includes any distinguishable manifestation of preeclampsia. For example and without limitation, the presence or absence of preeclampsia (diagnostic), the risk of developing preeclampsia (predictive) or the stage of preeclampsia.

The method of the invention may be used, for example, for any one of the following: to diagnose preeclampsia; to assess the chance of a subject developing preeclampsia, that is, to predict whether a subject is likely to develop preeclampsia; and to advise on the prognosis of a subject with preeclampsia.

The term ‘biological sample’ defined herein may refer to a sample of biological fluid obtained for the purpose of diagnosis or evaluation of a subject of interest. Preferred biological samples include, but are not limited to, blood, serum, and plasma. In addition, the person skilled in the art would realize that some test samples would be more readily analysed following a fractionation or purification procedure, for example, separation of whole blood into serum or plasma components. In another embodiment the biological sample may include exosomes isolated from a sample, such as a blood sample, provided by a subject. The biological sample may be a sample of placental tissue, preferably obtained postpartum.

In certain embodiments, the method of the invention may be performed on the biological sample without any prior major manipulation of the sample. In other embodiments, the inventive methods may be performed on nucleic acid extracts or protein extracts or exosomes prepared from the biological sample.

The step of obtaining the sample may not form part of the invention.

The subject may be a human.

Type XVII collagen alpha 1, encoded by COL17A1, is a transmembrane protein.

Leptin is a hormone predominantly produced by adipose cells.

Neprilysin is a zinc-dependent metalloprotease, also known as membrane metallo-endopeptidase (MME),

Filamin B is a cytoplasmic protein encoded by the FLNB gene.

Scavenger receptor class B member 1 is a membrane bound protein encoded by the SCARB1 gene

Accession numbers for the genes and proteins referred to above are given in the table below

| | Biomarkers | | | | |
|---|---|---|---|---|---|
| | | | | | Scavenger |
| Accession | | | | | receptor class |
| number | COL17A1 | Leptin | FLNB | neprilysin | B member 1 |
| Protein | NP_000485.3 | NP_000221.1 | NP_001157789.1 | NP_009220.2 | NP_001076428.1 |
| Gene | NM_000494.3 | NM_000230.3 | NM_001164317.1 | NM_000902.3 | NM_001082959.1 |

The presence of Type XVII collagen alpha 1 in a sample may be sufficient to be predictive or diagnostic of preeclampsia, there may be no need to determine the actual levels.

In an embodiment, if the level of Type XVII collagen alpha 1 in a plasma sample is at a concentration of about 10 ug/ml or more, this is diagnostic of preeclampsia.

In an embodiment, if the level of Type XVII collagen alpha 1 in a plasma sample is about 2 fold or more than the concentration in a control, this is diagnostic of preeclampsia.

In an embodiment, if the level of leptin in a plasma sample is at a concentration of about 50 ng/ml or more, or about 100 ng/ml or more, this is diagnostic of preeclampsia.

In an embodiment, if the level of leptin in a plasma sample is about 2 fold or more than the concentration in a control, this is diagnostic of preeclampsia.

In an embodiment, if the level of Filamin B in a plasma sample is at a concentration of about 50 ng/ml or more, this is diagnostic of preeclampsia.

In an embodiment, if the level of Filamin B in a plasma sample is about 2 fold or more than the concentration in a control, this is diagnostic of preeclampsia.

In an embodiment, if the level of neprilysin in a plasma sample is about 2 fold or more than the concentration in a control, this is diagnostic of preeclampsia.

In an embodiment, if the level of scavenger receptor class B member 1 in a plasma sample is about 2 fold or more than the concentration in a control, this is diagnostic of preeclampsia.

In addition to the specific biomarker sequences identified in this application by name or accession number, the invention also contemplates the detection in a test sample of naturally occurring variants that are at least 90%, or at least 95%, or at least 97%, identical to the exemplified biomarker sequences (either nucleotide or protein sequences). Said biomarker variants shall have utility for the methods of the invention and shall be detected via methods, as disclosed herein. These variants include but are not limited to polymorphisms, splice variants and mutations.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using a sequence comparison algorithm (e.g., BLASTP and BLASTN or other algorithms available to persons of skill) or by visual inspection. Depending on the application, the percent identity can exist over a region of the sequence being compared (e.g., over a functional domain) or, alternatively, exists over the full length of the two sequences to be compared.

The method of the invention may further comprise the step of comparing the level of biomarker expression determined in (b) with one or more reference values.

The reference value may be the level of one or more of Type XVII collagen alpha, leptin, neprilysin, Filamin B and Scavenger receptor class B member 1 in a sample obtained from a pregnant subject who does not have/does not go on to develop preeclampsia. Preferably the reference and sample tested are compared at the same time during a pregnancy, for example both relate to levels observed between about week 19 and about week 21 of pregnancy, or between about week 10 and about week 13 of pregnancy. Additionally or alternatively, the reference value may be a previous level of one or more of Type XVII collagen alpha, leptin, neprilysin, Filamin B and Scavenger receptor class B member 1 observed in the subject at an earlier time period, for example a value obtained from a sample taken about 10, 9, 8, 7, 6, 5, 4, 3, 2, or 1 weeks earlier.

Biomarker levels may be detected by determining protein levels in the sample, or by determining microRNA levels or mRNA levels in the sample.

Biomarker protein levels may be evaluated by any suitable method. For example, the level may be determined by immunoassays, spectrometry, western blot, ELISA, immunoprecipitation, slot or dot blot assay, isoelectric focusing, SDS-PAGE, antibody microarray immunohistological staining, radioimmunoassay (MA), fluoroimmunoassay, an immunoassay using an avidin-biotin or streptoavidin-biotin system, chromatographic techniques (i.e. immunoaffinity chromatography), flow cytometry etc. and combinations thereof. Other methods may also be used. These methods are well known to the person skilled in the art.

Biomarker levels may be determined by determining the expression level of genes encoding the biomarker protein. Nucleic acid-based techniques for assessing expression are well known in the art and include, for example, hybridization or amplification assays that include, but are not limited to, Southern or northern analyses, polymerase chain reaction analyses (PCR or reverse transcription polymerase chain reaction) and probe arrays. mRNA may be assayed directly, or it may be first transcribed into cDNA, which can then serve as template for multiple rounds of transcription by the appropriate RNA polymerase.

Biomarker levels may be determined on and/or in microvesicles isolated from maternal blood. Preferably microvesicles in maternal blood may be analysed when screening for leptin. The microvesicles may first be recovered from a maternal blood sample, preferably from a sample of platelet free plasma, prior to analysis. The microvesicles may be exosomes.

In a preferred embodiment levels of biomarkers are determined in a simple point of care test, perhaps with a colorimetric indicator on a spot test or lateral flow device.

Samples may be analyzed by means of a biochip. Biochips generally comprise solid substrates and have a generally planar surface to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art.

In a preferred embodiment the biomarker detection method is antibody based.

An advantage of the method of the invention is that it provides a good positive predictive value, that is it allows subjects to be identified who are likely to develop preeclampsia.

Whereas prior art tests have been good at identifying subjects who are unlikely to develop preeclampsia, but of those left it is not possible to predict who will develop preeclampsia. For example, one known test gives a 99% negative predictive value, but only a 36.8% positive predictive value, which is far from ideal in a clinical setting.

If a level of biomarker is observed that is predictive of preeclampsia early in pregnancy, say at 12 weeks, then a low dose of aspirin could be administered, say 75 to 150 mg/day which could reduce the chance of preeclampsia occurring by 30 to 50%. Alternatively, or additionally, if elevated neprilysin is observed then a neprilysin inhibitor, such as sacubitril, may be administered.

Early diagnosis of preeclampsia, and early intervention, could prevent early delivery of the baby and reduce the risks associated therewith.

The method of the invention may be used in conjunction with an assessment of clinical symptoms to assist in the diagnosis of preeclampsia.

According to another aspect of the invention, there is provided a method of treating preeclampsia in a pregnant subject, comprising:
(a) obtaining a biological sample from the subject;
(b) determining the level of one or more of Type XVII collagen alpha, leptin, neprilysin, Filamin B and Scavenger receptor class B member 1 in the biological sample; and
(c) administering anti-preeclampsia therapy to the subject if the level of one or more of Type XVII collagen alpha, leptin, neprilysin, Filamin B and Scavenger receptor class B member 1 in the sample is indicative and/or diagnostic and/or predictive of preeclampsia.

The anti-preeclampsia therapy may include, but is not limited to, the administration of aspirin and/or a neprilysin inhibitor.

According to another aspect of the invention, there is provided a kit for use in determining the preeclampsia status of a pregnant subject comprising at least one agent for determining the level of one or more of Type XVII collagen alpha, leptin, neprilysin, Filamin B and Scavenger receptor class B member 1, in a biological sample obtained from the subject.

The agent may be one or more antibodies. The agent may be one or more primer pairs.

The kit may comprise agents for detecting one biomarker. The kit may comprise agents for detecting two biomarkers. The kit may comprise agents for detecting three or more biomarkers.

For antibody-based kits, the kit may comprise, for example: (1) a first antibody (e.g., attached to a solid support) which binds a polypeptide corresponding to a biomarker of the invention; and, optionally (2) a second, different antibody that binds to either the polypeptide or the first antibody and is conjugated to a detectable label.

For oligonucleotide-based kits, the kit may comprise, for example: (1) an oligonucleotide, e.g., a detectably labeled oligonucleotide, which hybridizes to a nucleic acid sequence encoding a polypeptide corresponding to a biomarker of the invention, or (2) a pair of primers useful for amplifying a nucleic acid molecule corresponding to a biomarker of the invention.

The kit may further comprise instructions suitable for operational parameters in the form of a label or separate insert. The instructions may inform a consumer about how to collect the sample.

The kit may also comprise other components, such as a buffering agent, a preservative, a protein stabilizing agent, and/or components necessary for a detectable label. The kit may include reagents employed in the various methods, such as devices for withdrawing and handling blood samples, second stage antibodies, ELISA reagents, tubes, spin columns, and the like. Each component of the kit may be enclosed within an individual container and all of the various containers can be within a single package.

The kit may also comprise samples of one or more of Type XVII collagen alpha, leptin, neprilysin, Filamin B and Scavenger receptor class B member 1 to be used as standards for calibration and comparison. The kit may comprise instructions to compare the level of one or more of Type XVII collagen alpha, leptin, neprilysin, Filamin B and Scavenger receptor class B member 1 detected in a sample with a calibration sample or chart. The kit may also include instructions indicating what level of one or more of Type XVII collagen alpha, leptin, neprilysin, Filamin B and Scavenger receptor class B member 1 is diagnostic and/or predictive of preeclampsia.

According to a further aspect, the invention provides the use of the determination of the level of one or more of Type XVII collagen alpha, leptin, neprilysin, Filamin B and Scavenger receptor class B member 1, in a biological sample as a means of assessing the preeclampsia status in a pregnant subject.

According to a further aspect, the invention provides receiving identification that a pregnant subject is suffering from preeclampsia, and treating the subject by administering an anti-preeclampsia therapy. The identification may be provided by determining the level of one or more of Type XVII collagen alpha, leptin, neprilysin, Filamin B and Scavenger receptor class B member 1, in a blood sample, and comparing the level to a control value.

The invention may further provide a method of determining the risk of a woman developing eclampsia post-partum, the method comprising the determining the level of one or more of Type XVII collagen alpha, leptin, neprilysin, Filamin B and Scavenger receptor class B member 1 in a blood sample or placental sample obtained from a mother at delivery.

In a further aspect, the present invention provides a method of detecting the presence and/or level of one or more of Type XVII collagen alpha, leptin, neprilysin, Filamin B and Scavenger receptor class B member 1, said method comprising
(a) providing a biological sample obtained from the subject; and
(b) detecting whether one or more of Type XVII collagen alpha 1, leptin, neprilysin, Filamin B and Scavenger receptor class B member 1 is in the biological sample by contacting the sample with an anti-Type XVII collagen alpha 1 antibody, an anti-leptin antibody, an anti-neprilysin antibody, an anti-Filamin B antibody and/or an anti-Scavenger receptor class B member 1 antibody and detecting binding between one or more of the anti-Type XVII collagen alpha 1 antibody and Type XVII collagen alpha 1, the anti-leptin antibody and leptin, the anti-neprilysin antibody and neprilysin, the anti-Filamin B antibody and Filamin B and/or the anti-Scavenger receptor class B member 1 antibody and Scavenger receptor class B member 1.

In another aspect the invention provides a method of diagnosing and treating preeclampsia in a pregnant subject, the method comprising:
(a) providing a biological sample obtained from the subject;
(b) determining the presence, and/or level, of Type XVII collagen alpha 1 in the biological sample;
(c) diagnosing the subject with preeclampsia when the presence of Type XVII collagen alpha 1 in the sample is detected; and
(d) administering an effective amount of aspirin to the diagnosed patient.

In a yet further aspect the invention provides a method of selecting a subject for treatment for preeclampsia, the method comprising:
(a) providing a biological sample obtained from the subject;
(b) analyzing the sample for the presence, and/or level, of one or more of Type XVII collagen alpha 1, leptin, neprilysin, Filamin B and Scavenger receptor class B member 1 in the biological sample;
(c) if one or more of Type XVII collagen alpha 1, leptin, neprilysin, Filamin B and Scavenger receptor class B member 1 are elevated relative to a reference control, selecting the subject treatment for preeclampsia.

In an embodiment step (c) may only require the presence of Type XVII collagen alpha 1 in order for a subject to be selected for treatment for preeclampsia.

The skilled person will appreciate that preferred features of any one embodiment and/or aspect of the invention may be applied to all other embodiments and/or aspects of the invention.

There now follows by way of example only a detailed description of the present invention with reference to the accompanying drawings, in which:

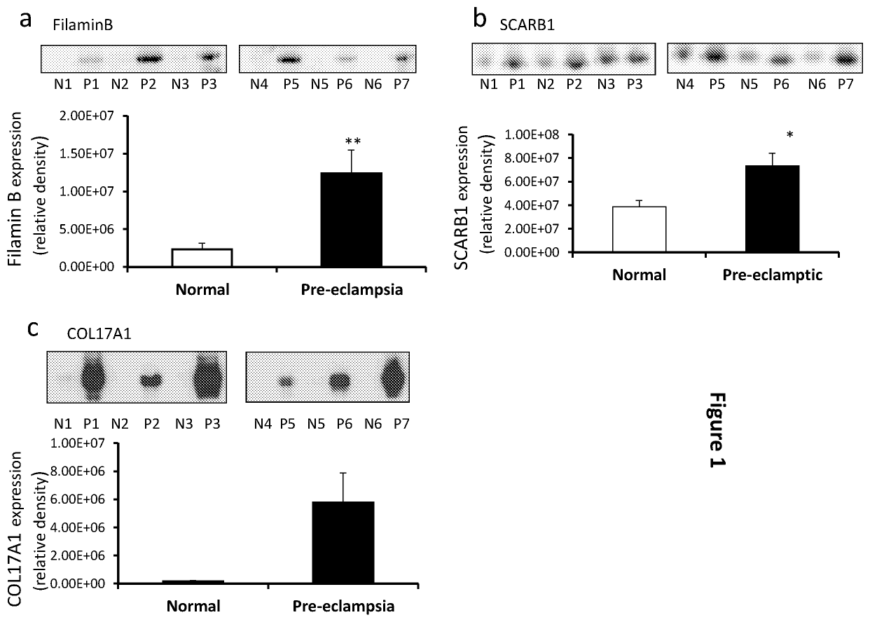

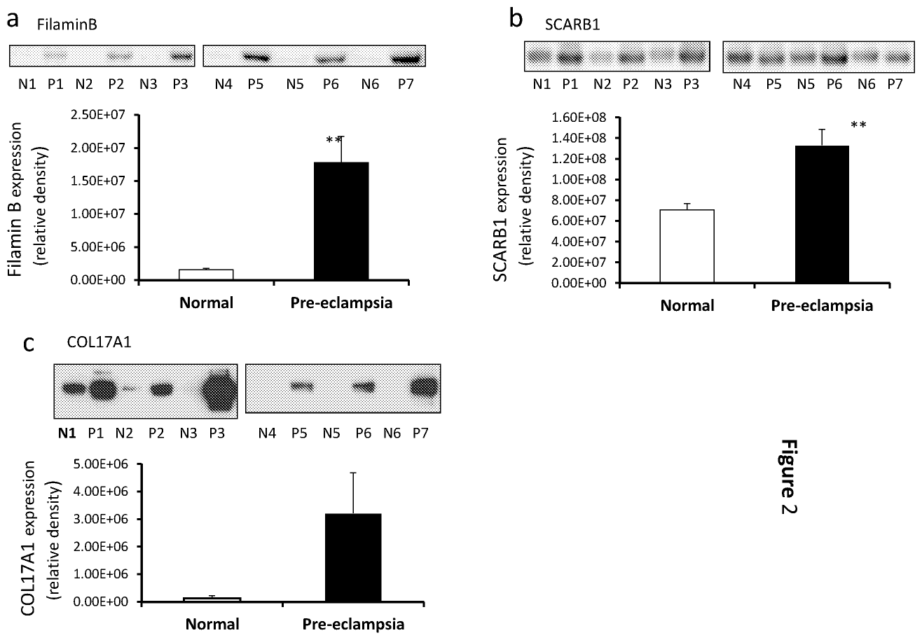

FIG. 1 shows analysis of the level expression of each of Type XVII collagen alpha (FIG. 1c), Filamin B (FIG. 1a) and Scavenger receptor class B member 1 (FIG. 1c) in microvesicles obtained from placental tissue of mothers with and without preeclampsia. N=without preeclampsia; and P=with preeclampsia, and for each state 7 subjects were compared. The data is presented by way of a Western Blot and then graphical comparing the relative density of the bands on the western blot.

FIG. 2 shows analysis of the level expression of each of Type XVII collagen alpha (FIG. 1c), Filamin B (FIG. 1a) and Scavenger receptor class B member 1 (FIG. 1c) in exosomes obtained from placental tissue of mothers with and without preeclampsia. N=without preeclampsia; and P=with preeclampsia and for each state 7 subjects were compared. The data is presented by way of a Western Blot and then graphical comparing the relative density of the bands on the western blot.

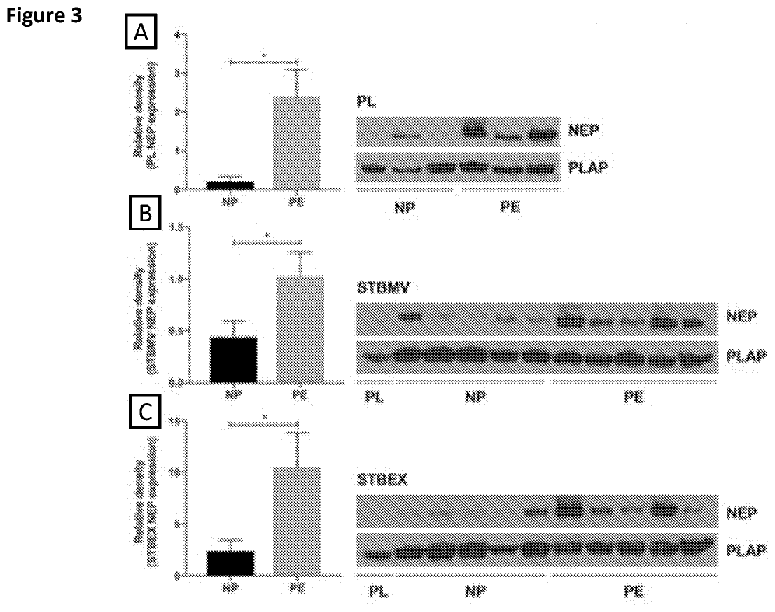

FIG. 3 shows analysis of the level expression of neprilysin (NEP) in placenta lysate (PL) (FIG. 3a), in placenta perfusion derived microvesicles (STBMV—syncytiotrophoblast extracellular vesicles) (FIG. 3b) and exosome fractions (STBEX—syncytiotrophoblast extracellular exosomes) (FIG. 3c). NP=without preeclampsia; and PE=with preeclampsia and for each state 7 subjects were compared. The data is presented by way of a Western Blot and then graphical comparing the relative density of the bands on the western blot. PLAP is used as loading control.

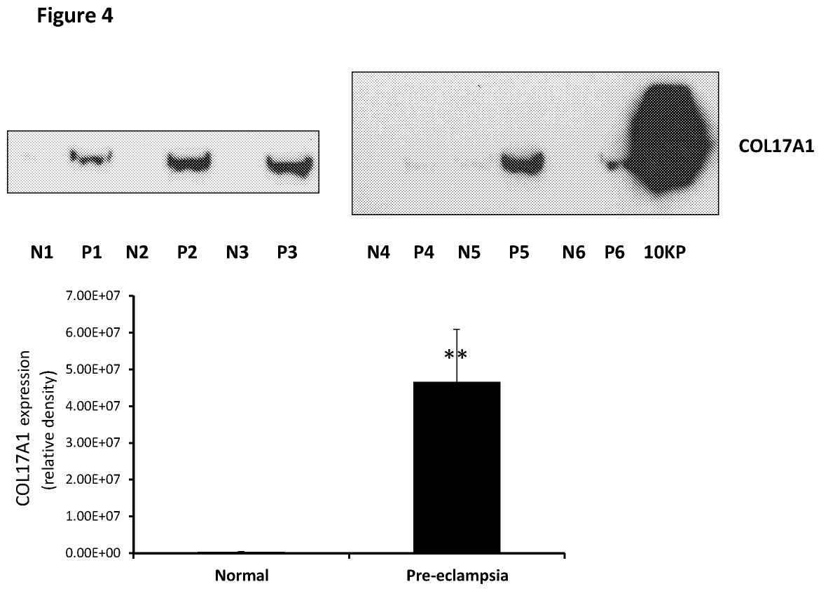

FIG. 4 shows analysis of the level expression of Type XVII collagen alpha in blood samples obtained from pregnant women with and without preeclampsia in the third trimester of pregnancy. N=without preeclampsia; and P=with preeclampsia and for each state 6 subjects were compared. The data is presented by way of a Western Blot and then graphical comparing the relative density of the bands on the western blot.

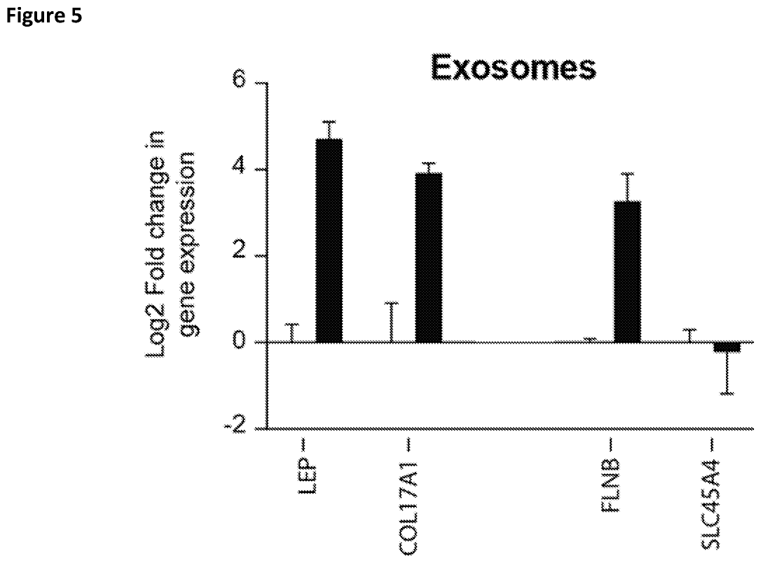

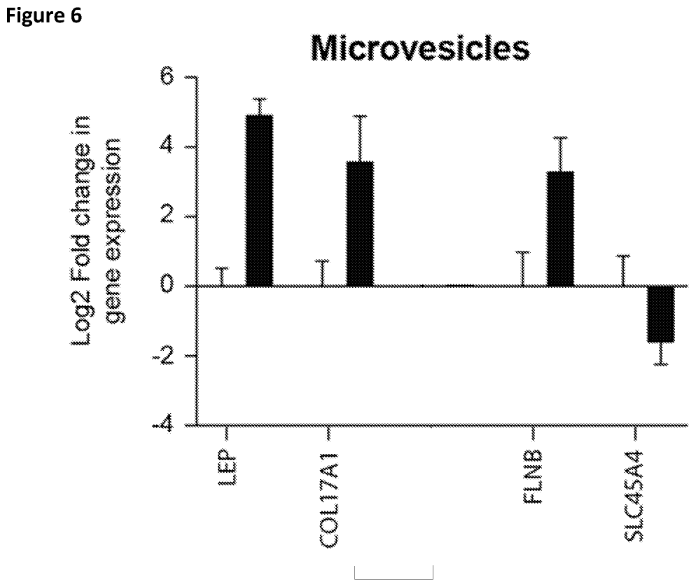

FIG. 5 and FIG. 6 shows analysis of the level of expression of each of LEP, COL17A1, FLNB and SLC45A4 in placenta perfusion derived exosomes and microvesicles respectively obtained from pregnant women with preeclampsia. The data presented illustrates the fold increase or decrease compared to normal.

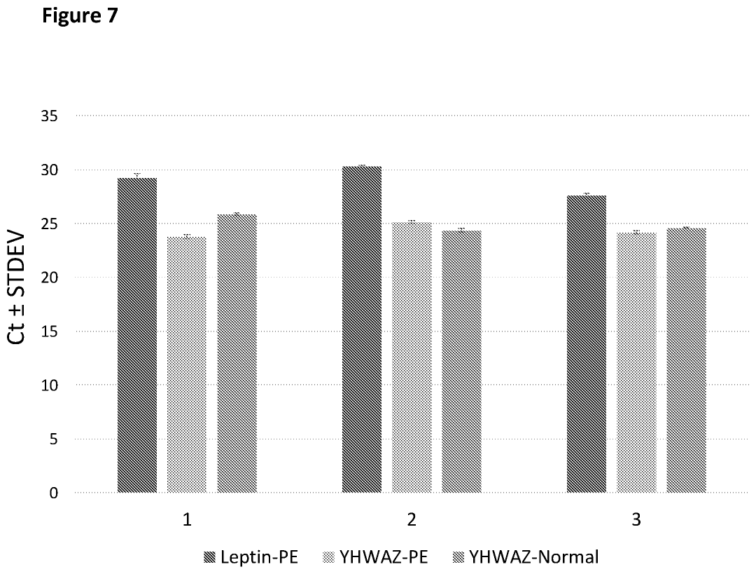

FIG. 7 shows the detection of leptin gene expression in EV (extracellular vesicles) isolated from plasma samples by RT-PCR. Housekeeping gene YHWAZ is detected in both patients and three gestation age matched normal controls. Leptin mRNA was only detected in PE (preeclampsia) samples.

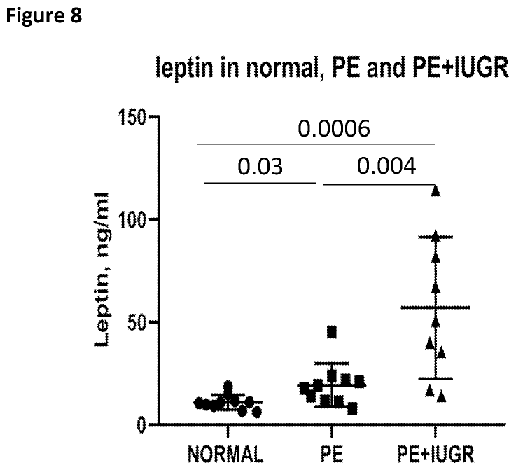

FIG. 8 shows level of leptin protein in patients' peripheral blood serum samples analyzed by ELISA. Leptin is significantly higher in PE (p=0.03) and further elevated in PE with intra-uterine growth restriction (IUGR) (p=0.004).

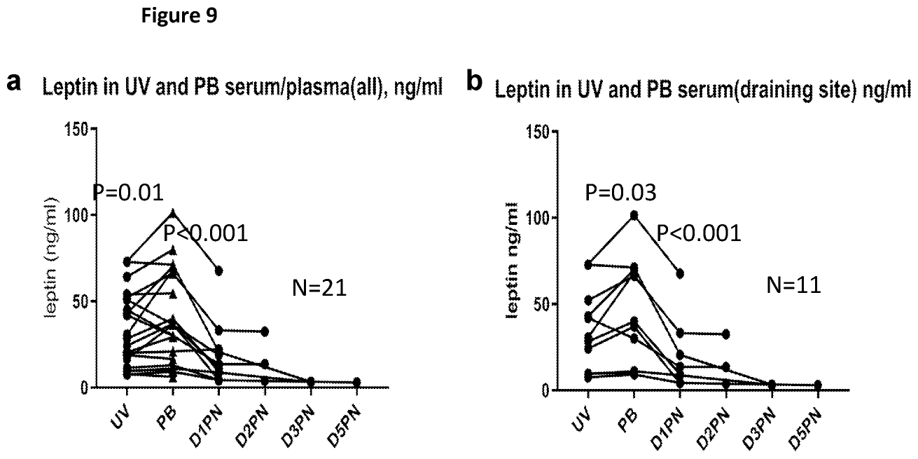

FIG. 9 shows level of leptin protein analyzed by ELISA in patients' peripheral blood serum samples and compared to their uterine vein samples (UV). Peripheral blood serum samples taken before placental removal (PB), on different day postpartum (D1PN, D2PN, D3PN, D5PN). Delivery of placenta fetal units saw a sharp decrease in leptin on the D1PN samples (p<0.001). Levels of leptin continue to fall during the subsequent postpartum days. Leptin is significantly higher (p=0.01) in the PB than the UV from all patients analyzed (FIG. 9a) and compared to UV from the placenta draining sites (FIG. 9b).

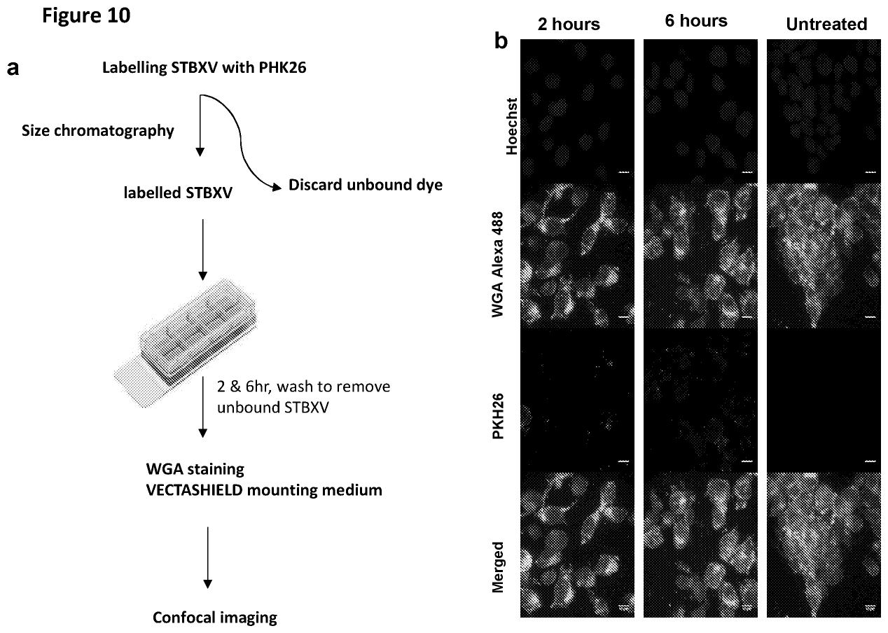

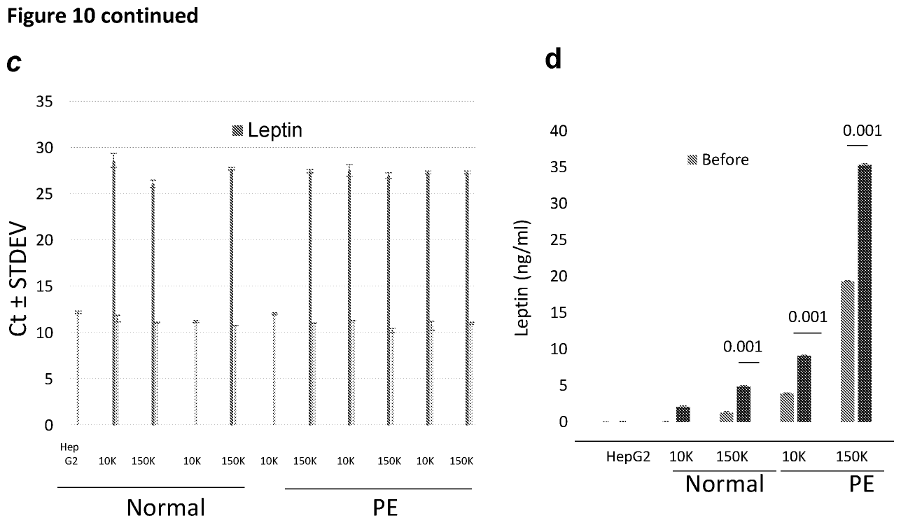

FIG. 10 shows leptin gene expression and leptin secretion by STB-MV and STB-XV treated HepG2 cells. FIG. 10A is a schematic workflow of HepG2 cells co-cultured with PHH26 labelled STBXV. FIG. 10B shows uptake of PKH26 stained STBXV by HepG2 cells; FIG. 10C shows rt-PCR for detection of leptin mRNA. Leptin mRNA not detected in HepG2 cells. Leptin mRNA was detected in HepG2 cells treated with 150K from both normal and PE. Leptin mRNA was detected in one out of two normal 10 k and two out of three PE 10K treated HepG2 cells; housekeeping gene YHWAZ, was detected in HepG2 cells of all culture conditions. Data is a representative of three experiments. FIG. 10D shows leptin in medium of HepG2 cultures+/−EVs. STB-MV and STB-XV pooled from three normal and PE patients were used in each condition. Before culturing with HepG2, leptin in medium, STB-MV and STB-XV each at 50 ug/ml from normal and PE (light grey bar); after culturing with HepG2, leptin in medium, STB-MV and STB-XV from normal and PE (dark bar).

### METHODS

### Human Subjects

Approval for this project was granted by the Central Oxfordshire Research Ethics Committee (07/H0607/74 and 07/H0606/148). Women recruited for this study provided written informed consent. According to the criteria defined by the International Society for the Study of Hypertension in Pregnancy (ISSHP), de novo hypertension (blood pressure >140/90 mmHg) and proteinuria (>300 mg/day) after gestational week 20 was used to diagnose PE. Women with normal term pregnancy were identified as those with singleton pregnancies and no background of PE or hypertensive disorders.

### STBMV and STBEX Isolation and Purification

Placental perfusate from a dual placental lobe perfusion system was centrifuged to pellet fractions containing STBMV (10 000×g) and STBEX (150 000×g). The method has previously been described in detail by Dragovic et al Methods. 2015 Oct. 1; 87:64-74. The protein concentration of isolated STBMV and STBEX was determined by the BCA protein assay before freezing.

Peripheral blood samples were taken from the left antecubital fossa and collected in 4.5 mL citrate vacutainer tubes using a 21-gauge needle. Samples were centrifuged at 1 500×g for 15 minutes and the Platelet poor plasma (PPP) was collected. Aliquots of 1 mL PPP were stored at −80° C. in preparation for flow cytometry analysis. Half of the PPP was further centrifuge at 13000 g for 2 minutes, the supernatant which is the platelet free plasma (PFP) was prepared for extracellular vesicles (EV) isolation, WB and ELISA analysis. Plasma samples collected from women with PE (preeclampsia) and NP (no preeclampsia) were matched according to gestational age. Placentas from elective caesarean section were collected within 10 minutes of delivery and subjected to dual placental lobe perfusion.

### Western Blotting

Western blotting was performed using placental lysate (PL, 18 μg) and STBEV (6 μg) from NP and PE placentas. STBMV and STBEX were treated with HEPES lysis buffer (1:1). Western blots were incubated overnight at 4° C. with either mouse monoclonal anti-NEP antibody (1:1000, ab951; Abcam), anti-FLNB (GT387, GenTex), antiCOL17A1 (ab184996, Abeam) and mouse monoclonal anti-placental alkaline phosphatase (PLAP) antibody (1:1000; NDOG2, in-house antibody). After incubation with HRP-conjugated anti-mouse IgG antibody (1:4000) for 1 hour at room temperature, Pierce™ ECL Western Blotting Substrate (Thermo Fisher Scientific) was used for chemiluminescence detection of antigens on x-ray film. Total protein normalization of the Western blots was completed using Coomassie Brilliant Blue R-250 (Bio-Rad Laboratories, California, USA) and ImageJ version 1.51 (public domain).

Purification of Exosomes from PFP Samples

Exosomes were purified from PFP (platelet free plasma) samples from PE patients and gestation matched normal controls using qEVsingle size exclusion columns (IZon Science) according to the manufacturer's instructions. Plasma (150 μl) was loaded onto a rinsed column and the void volume (1000 μl) was collected and discarded. The vesicle fraction, contained in the next 600 μl, was then collected in a fresh tube. This was then concentrated to a volume of 100 μl using an Amicon Ultra-4 centrifugal filter unit before RNA isolation.

### Purification of RNA

Total RNA was isolated using miRCURY™ RNA Isolation Kits (Exiqon, Manchester, UK) from 10K, 150K pellets, and frozen placenta tissues of both normal and PE patients. RNA was also isolated from exosomes isolated from PFP samples. Genomic DNA was removed using the Precision DNase kits (Primer Design, UK). Purity and concentration of isolated total RNA was measured using NanoDrop® ND-1000 UV-Vis spectrophotometer (Thermo Fisher Scientific Inc., UK) before further downstream analysis. Additional quality controls were performed before NGS by the sequencing facility at the Welcome Human Genomic Centre, Oxford.

### Reverse Transcription and PCR

cDNA was synthesised with High Capacity cDNA Reverse Transcription Kits (Thermo Fisher Scientific, UK) following the manufacturer's protocol using a G-Strom thermocycler (G-Storm, Somerset, UK). TaqMan Gene Expression Assays were used for PCR quantitation of the differentially expressed genes in all samples following manufacture's protocol (Thermo Fisher Scientific, UK). YWHAZ (Hs01122445_g1) was used as an endogenous control since it was found to be stably expressed in all samples tested in this study. Gene expression studies of COL17A1 (Hs00990036_m1), Leptin (Hs00174877_m1), Siglec 6 (Hs00609663_m1), Filamin-B (Hs00963204_m1) were conducted in triplicate using a Rotor-Gene Q 2× platform. Fold changes in expression were calculated following the published method. No template controls were used as negative controls in each run.

### Culture of HepG2 Cells

HepG2 cells obtained from the ATCC were cultured in RPMI-1640 supplemented with 10% (v/v) of foetal bovine serum (FBS) and 1% (v/v) of antibiotics solution (all from Sigma) placed in a humidified incubator at 37° C. and 5% CO2. Cultures were passaged when cell density reached 80-90% confluence.

### Vesicle Treatment

HepG2 cells were seeded into a 24-well-plate at 5×10⁴/ml in RPMI 1640 supplemented with 10% FBS. Culture medium was removed 24 hr later and the cells were then washed three times with warm PBS to remove residual FBS before adding 0.5 ml of RPMI-1640 supplemented with 1% (v/v) Panexin basic serum replacement (PAN Biotech), 1% (v/v) antibiotics, and 1% antimycotic to each well. For measurement of leptin production, 10K and 150K preparations, each pooled from three normal and three PET patients were added to HepaG2 cultures at 25 ug/ml for two consecutive days. Equal volume of PBS used to resuspend the 10K and 150K pellet was added to the control cultures. At the end of 48 hr treatment period, culture supernatant was collected and spun at 1500 g for 5 minutes to remove cells before the cell free supernatant was frozen for leptin ELISA. For detection of leptin mRNA in HepG2 cells, 50 ug/ml of 10K and 150K from individual normal and PE patients were added to HepG2 cultures. After 24 hr treatment, cells were washed with warm PBS to remove unbound vesicles before harvested and freezing for RNA isolation and gene expression analysis.

### Leptin ELISA

Leptin in serum, plasma, and medium spiked with either 10K or 150K samples was quantified using the Leptin Quantikine ELISA kits (Sensitivity 7.8 pg/ml) (R&DSystem, Milton Park, UK). Serum or plasma samples were used at 1 in 200-400 dilution. Leptin in 10K, 150K from normal and PE patients were measured by diluting these samples in RPMI 1640 to a total protein concentration of 50 ug/ml. Culture supernatant from HepG2 treated with medium alone or 10K or 150K, both at 50 ug/ml, from both normal and PE patients was used for leptin production measured.

Immunofluorescence with PKH26 Stained Syncytiotrophoblast Extracellular Vesicles (STBEX)

50 μg of pooled normal pregnancy STBEX were stained using the PKH26 red fluorescent cell linker kit (MINI26, Sigma) according to manufacturer's instructions. Briefly, STBEX were diluted in 200 μL of Diluent C and mixed with 200 μL of Diluent C and 1.6 μL of PKH26 stock (1 mM). The mix was left to incubate for 5 minutes in the dark and the reaction was stopped by 1 minute incubation with 400 μL of 1% BSA in PBS in the dark.

Next, stained exosomes were washed thoroughly using the qEV size exclusion columns (Izon Science) following manufacturer's protocol. Briefly, columns were washed with 30 mL of filtered PBS prior to use. Stained exosome sample was carefully added to the column and 12 fractions of 500 μL in volume were collected thereafter. The fractions were assessed by nanoparticle tracking analysis (NTA) to confirm size and particle number per fraction. Fractions 7-10 were pooled together and STBEX were concentrated in a 50 μL volume using an Amicon ultra-4 centrifugal filter unit with ultracel-100 membrane (Merck Millipore). To remove PKH26, stained STBMV were washed by ultracentrifugation at 10,000×g for 35 minutes and resuspended in 50 μL filtered PBS.

Final stained STBEV were added to an initial volume (500 μL) of serum free media and particle number was assessed by NTA. Each sample was diluted accordingly to achieve the same particle number among sample groups. Cells at a concentration of 2.5×10⁵ cells/mL were grown overnight on autoclaved coverslips in a 24-well plate. The next day, cells were washed with warm PBS and incubated with extracellular vesicle (EV)-free media and the corresponding stained EVs for 2 and 6 hours. After incubation, cells were carefully and thoroughly washed with warm PBS. Cells were then incubated with 5 μg/mL of wheat germ-like agglutinin (WGA) green (Vector Laboratories) diluted in media for 5 minutes at room temperature in dark. Cells were washed three times with PBS followed by an incubation of 10 minutes with 2% PFA. Again, cells were thoroughly washed. Next, cells were stained with 1/5,000 Hoechst 33342 (Thermofisher) for 5 minutes. Finally, cells were thoroughly washed and coverslips were carefully moved with tweezers to slides, cells facing upwards. Slides were mounted with 0.1 μm coverslips using fluorescent mounting medium (Vectashield).

Cells were viewed under a Zeiss confocal microscope with Yokogawa spinning disk scanning unit and an attached Evolve® 512 Delta EMCCD camera, and images were taken using a Zen Blue software (Zeiss). Z-stacks (11 slices) were taken through a 10 μm width and images were compressed on maximum intensity using ImageJ software (Fiji).

### RESULTS AND CONCLUSION

The data presented herein clearly shows that in a subject with preeclampsia the levels of at least one of filamin B, COL17A1, Leptin, neprilysin and SCARB1 are elevated.

More specifically, the data presented in FIGS. 1a, 1b and 1c clearly demonstrates that expression of Type XVII collagen alpha, Filamin B and Scavenger receptor class B 1 is elevated in microvesicles obtained from the placental tissue of mothers with preeclampsia, compared to those without preeclampsia. The data presented demonstrates that Type XVII collagen alpha, Filamin B and Scavenger receptor class B member 1 are biomarkers for preeclampsia.

The data presented in FIGS. 2a, 2b and 2c clearly demonstrate that expression of Type XVII collagen alpha, Filamin B and Scavenger receptor class B member 1 is elevated in exosomes obtained from the placental tissue of mothers with preeclampsia, compared to those without preeclampsia. The data presented demonstrates that Type XVII collagen alpha, Filamin B and Scavenger receptor class B member 1 are biomarkers for preeclampsia.

The data presented in FIG. 3 clearly demonstrates that expression of neprilysin is elevated in the placental tissue, microvesicles and exosomes obtained from the placenta of mothers with preeclampsia, compared to those without preeclampsia. Neprilysin is therefore a biomarker for preeclampsia.

Consistent with RNAseq differential expression of Leptin, COL17A1, FLNB and SLC45A4 was confirmed by rt-PCR data (FIGS. 5 and 6) in both microvesicle and exosome samples from placenta perfusion. Further, leptin mRNA was only detected in exosomes obtained from the plasma of subjects with early-onset pre-eclamptic toxaemia (PET) plasma but not in the gestation matched normal controls. Housekeeping gene YHWAZ was detected in all samples (FIG. 7). Thereby demonstrating that exosomes in a subjects plasma may be used to diagnose preeclampsia based on the leptin mRNA they carry.

Leptin was found to be significantly elevated in serum and plasma samples from PET patents compared to normal controls (p=0.03). The levels of serum and plasma leptin was highly significantly elevated in early-onset preeclampsia with intrauterine growth restriction (IUGR) compared to normal controls (P=0.0006) and PET patients (p=0.004) (FIG. 8). The data presented indicates that elevated serum and plasma leptin levels associate with disease severity. Combining the detection of leptin protein and plasma exosomal leptin mRNA increases specificity in early onset PET+IUGR diagnosis.

Consistent with published data, there was a continuous decline of serum leptin in postnatal peripheral blood (PB) from all patients' samples tested with the sharpest drop occurring on day one postnatal samples (p<0.001, n=). In contrast, levels of leptin were significantly lower in the uterine vein (UV) compared to the paired PB (all, p=0.01, n=21, FIG. 9a; UV from the placental draining side, p=0.03, n=11, FIG. 9b). Taken together, this data supports the conclusion that the placenta foetal unit drives pregnancy related maternal leptin production. The placenta foetal unit itself however is not the major leptin producer.

Studies were undertaken to determine whether leptin mRNA carried by microvesicles and exosomes can be transferred to cells (HepG2 cells) which lack endogenous leptin transcription and translation. The results showed that PKH26 labelled 150K due enter HepG2 cells (FIG. 10b). The results also showed that leptin transcription was absent in untreated HepG2 cells but leptin mRNA became detectable in all 150K treated HepG2 samples, and three out of five 10K sample treated HepG2 cells (FIG. 10c). Leptin was detected in 10K and 150K samples from both the normal controls and the PET patients with the highest levels of leptin found in the PET patients' exosome samples (FIG. 10d).

Treatment of HepG2 cells with 10K and 150K from both normal and PET patients induced a significant increase in leptin in the culture medium. Leptin was absent in the culture medium before and after HepG2 culture (FIG. 10D). The data shows that leptin translation occurs in HepG2 cells after treatment with placental EVs. The treatment of HepG2 cells with STBEX or STBMV induced leptin production by HepG2 cells. Production of leptin was further elevated by HepG2 treatment with STBEX from PE patients. These results demonstrate that the placenta foetal unit drives maternal leptin production through the release of STBEX. In addition to soluble leptin, leptin that is physically associated with the microvesicle and exosome may be a biomarker for preeclampsia.

1. A method of determining the preeclampsia status of a pregnant subject, comprising:
(a) providing a biological sample obtained from the subject; and
(b) determining the presence or absence, and/or level, of one or more of Type XVII collagen alpha 1, leptin, neprilysin, Filamin B and Scavenger receptor class B member 1 in the biological sample.

2. The method of claim 1 wherein in step (b), at least the presence, absence and/or level of Type XVII collagen is determined.

3. The method of any preceding claim for one or more of diagnosing preeclampsia; assessing the chance of a subject developing preeclampsia; and advising on the prognosis of a subject with preeclampsia.

4. The method of any preceding claim wherein the biological sample is blood, serum, or plasma.

5. The method of claim 4 wherein the biological sample comprises exosomes isolated from a sample, such as a blood sample, provided by a subject.

6. The method of any preceding claim wherein the level of one or more of Type XVII collagen alpha 1, leptin, neprilysin, Filamin B and Scavenger receptor class B member 1 is determined at the nucleic acid level and/or at the protein level.

7. The method of any preceding claim wherein the presence of Type XVII collagen alpha 1 in a sample is predictive or diagnostic of preeclampsia.

8. The method of any preceding claim wherein if the level of Type XVII collagen alpha 1 in a plasma sample is at a concentration of about 1 ng/ml or more, this is diagnostic of preeclampsia.

9. The method of any preceding claim wherein if the level of Type XVII collagen alpha 1 in a plasma sample is about 2 fold or more than the concentration in a control, this is diagnostic of preeclampsia.

10. The method of any preceding claim wherein if the level of leptin in a plasma sample is at a concentration of about 100 ng/ml or more, this is diagnostic of preeclampsia.

11. The method of any preceding claim wherein if the level of leptin in a plasma sample is about 2 fold or more than the concentration in a control, this is diagnostic of preeclampsia.

12. The method of any preceding claim wherein if the level of Filamin B in a plasma sample is at a concentration of about 50 ng/ml or more, this is diagnostic of preeclampsia.

13. The method of any preceding claim wherein if the level of Filamin B in a plasma sample is about 2 fold or more than the concentration in a control, this is diagnostic of preeclampsia.

14. The method of any preceding claim further comprising the step of comparing the level of biomarker expression determined in (b) with one or more reference values.

15. A method of treating preeclampsia in a pregnant subject, comprising:
(a) obtaining a biological sample from the subject;
(b) determining the level of one or more of Type XVII collagen alpha, leptin, neprilysin, Filamin B and Scavenger receptor class B member 1 in the biological sample; and
(c) administering anti-preeclampsia therapy to the subject if the level of one or more of Type XVII collagen alpha, leptin, neprilysin, Filamin B and Scavenger receptor class B member 1 in the sample is indicative and/or diagnostic and/or predictive of preeclampsia.

16. The method of claim 15 wherein the anti-preeclampsia therapy may is the administration of aspirin or a neprilysin inhibitor.

17. A kit for use in determining the preeclampsia status of a pregnant subject comprising at least one agent for determining the level of one or more of Type XVII collagen alpha, leptin, neprilysin, Filamin B and Scavenger receptor class B member 1, in a biological sample obtained from the subject.

18. The kit of claim 18 comprising agents for detecting two or more biomarkers.

19. The kit of claim 17 or claim 18 wherein the agent is an antibody or wherein the agent is an oligonucleotide-based kits.

20. The kit of any of claims 17 to 18 further comprising other components, including one or more of a buffering agent, a preservative, a protein stabilizing agent, and/or components necessary for a detectable label.

21. The use of the determination of the level of one or more of Type XVII collagen alpha, leptin, neprilysin, Filamin B and Scavenger receptor class B member 1, in a biological sample as a means of assessing the preeclampsia status in a pregnant subject.

22. A method of determining the risk of a woman developing eclampsia post-partum, the method comprising the determining the level of one or more of Type XVII collagen alpha, leptin, neprilysin, Filamin B and Scavenger receptor class B member 1 in a blood sample or placental sample obtained from a mother at delivery or within 1 hour of birth.

23. A method of diagnosing and treating preeclampsia in a pregnant subject, the method comprising:
(a) providing a biological sample obtained from the subject;
(b) determining the presence, and/or level, of Type XVII collagen alpha 1 in the biological sample;
(c) diagnosing the subject with preeclampsia when the presence of Type XVII collagen alpha 1 in the sample is detected; and
(d) administering an effective amount of aspirin to the diagnosed patient.

24. A method of selecting a subject for treatment for preeclampsia, the method comprising:
(a) providing a biological sample obtained from the subject;
(b) analyzing the sample for the presence, and/or level, of one or more of Type XVII collagen alpha 1, leptin, neprilysin, Filamin B and Scavenger receptor class B member 1 in the biological sample;
(c) if one or more of Type XVII collagen alpha 1, leptin, neprilysin, Filamin B and Scavenger receptor class B member 1 are elevated relative to a reference control, selecting the subject treatment for preeclampsia.
